(19) United States
(12) Patent Application Publication
(10) Pub. No.: US 2018/0005335 A1
(43) Pub. Date: Jan. 4, 2018

(54) VIDEO DISPOSITION INSTRUCTIONS

(71) Applicants: John Parente, Avon, OH (US); Jansen Musselman, Westlake, OH (US); Devin Hale, Medina, OH (US)

(72) Inventors: John Parente, Avon, OH (US); Jansen Musselman, Westlake, OH (US); Devin Hale, Medina, OH (US)

(21) Appl. No.: 15/491,578

(22) Filed: Apr. 19, 2017

Related U.S. Application Data
(60) Provisional application No. 62/324,591, filed on Apr. 19, 2016.

(51) Int. Cl. G06Q 50/24; A61B 5/00; G06F 17/30; G06F 19/00

(52) U.S. Cl. CPC G06Q 50/24; G06F 19/34; A61B 5/74; G06F 17/30876

(57) ABSTRACT

One or more techniques and/or systems are disclosed for a medical disposition process for use in medical treatment, in such locations as hospital, ERs, medical clinics, and other places where patients may be treated. The disclosed disposition process can also be used during patient services, and may be able to help the patient improve the outcome of a medical condition.

### BACKGROUND

Healthcare clinician visits constitute a common interaction between the clinician and patient, and may include routine check-ups, sicknesses or injuries, surgical procedures, and emergency room (ER) visits. ER visits alone constitute over 135 million patient encounters per year. The disposition process includes, but is not limited to, pre-visit admission, after visit discharge, after stay discharge, ER discharge, ER admission, observation, inpatient status, home health, and nursing home placement. The disposition process is not standardized and can vary in different facilities.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

In one implementation, a standardized medical disposition process is provided. The disposition process can include video or other media disposition instructions. The standardized disposition process may cover patient visit for medical consultations, treatments or diagnosis, and other places where patients may be treated. A standardized disposition process may also cover other service lines and other disciplines such as patient input, output, and post-operative information.

In one implementation, a method for medical disposition of a patient can comprise producing media content comprising patient disposition information specific to a medical condition associated with a patient. In this implementation, the media content can instruct the patient regarding steps to take to improve outcome for the medical condition. Further, for example, method can comprise loading the media content to a platform that is accessible by the patient or patient's guardian. Additionally, the media content can be automatically provided on a display accessible by the patient or patient's guardian upon the platform receiving an activation signal.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

What is disclosed herein may take physical form in certain parts and arrangement of parts, and will be described in detail in this specification and illustrated in the accompanying drawings which form a part hereof and wherein:

FIG. 1 is a flow diagram of and exemplary method for medical disposition.

FIG. 2 is a schematic diagram illustrating and example implementation of a system for medical disposition

FIG. 3 is a component diagram illustrating one or more portions of one or more systems described herein.

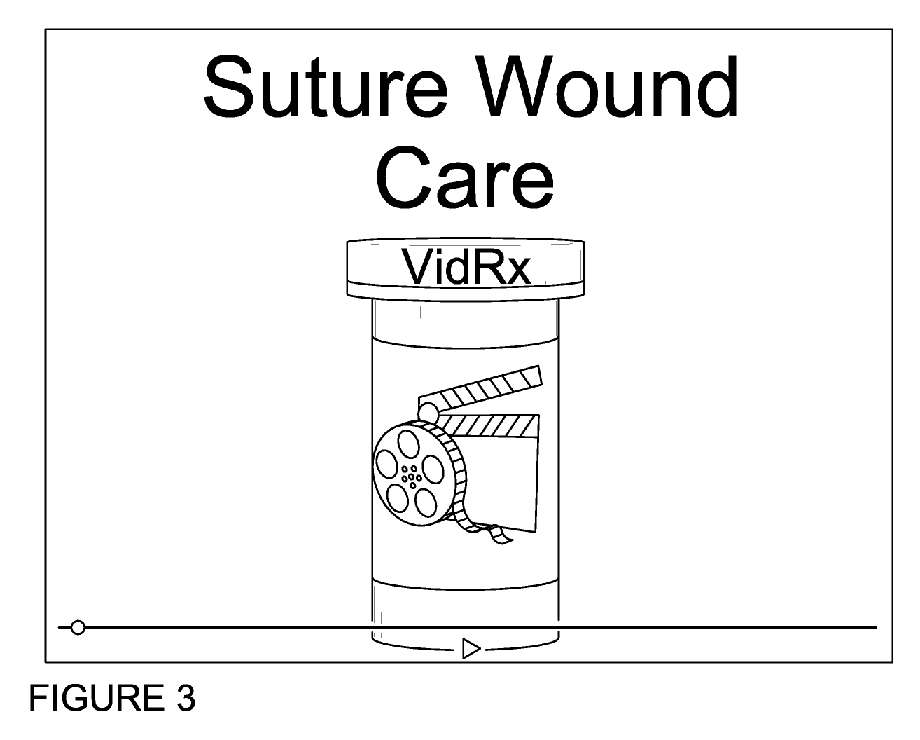

FIG. 4 is a component diagram illustrating one or more portions of one or more systems described herein.

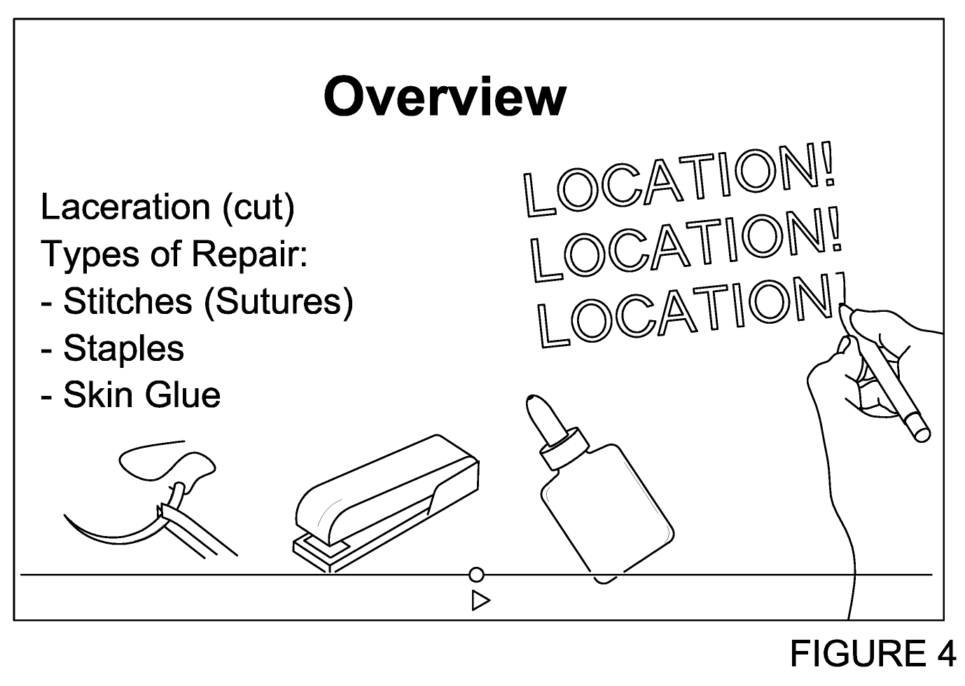

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

The disposition includes medical information for a patient that can provide information for a patient before or after medical treatment such as surgery or other patient visits. The disposition may include a follow-up list detailed in the health care record, after the initial episode of care, of services and treatments to be provided to the patient, including medication information. Currently, the disposition is not standardized at different facilities or wherever patients are treated. A standardization of the disposition process, for example in the ER discharge, ER admission, observation, inpatient status, home health, and nursing home placement, may provide consistency to the process, especially where patients are treated in multiple locations. A standardization of the disposition process may allow: 1) reduced medicolegal exposure by standardizing verbiage; 2) improved communication regarding disposition instructions, including helping those patients/families with illiteracy, visually impaired and patients/families with language barriers; 3) improve employee morale (physicians, APC's, RN's, etc.) since it will allow clinicians to function more efficiently; and 4) improve metrics (Decreased: LOS, LWOT, D2Discharge, Preventable readmit rates; Increased: PG scores, EDCAHPS, HCAHPS).

In order to provide the standardized disposition process as disclosed, a software platform, app, and/or video component may be used. In one implementation, a web based platform may be provided to launch disposition media, such as a video, multimedia presentation, animated media, interactive content, web-based application, or combinations of the same. In another implementation, an app on a health care provider or patient's phone may be used in the disposition process. In another implementation, a clinician may activate a web-based link, such as on a local device, as part of the disposition process. For example, the link may be a part of the video system in the room, or on a computer provided. In one implementation, videos for the disposition process may be provided on jump drives, optical drive discs, online video hosting services, hospital/clinic networks or websites, in room computing devices, other hardware (e.g. specifically portable). For example, if the clinic does not have mobile computers or has limited wireless capabilities, personal or other devices may be used, or any other medium that may provide access to the media content.

Additionally, a standardized disposition method and system, as described herein, may provide media content for patients who have common diagnoses or visit issues, such when discharged from an ER, with the ability to expand the information to patients who are admitted for stays at a hospital or clinic. As an example, the media content may comprise explanations for certain tests or diagnostic tools, such as an Mill, colonoscopy, EKG, cardiac catheterization, and stress tests, as well as certain surgical procedures. In another implementation, a patient portal comprising the patient's healthcare information may be provided for use through an online application, mobile app, or other web based platform. In yet another implementation, a standardized disposition method or system, as described herein, may comprise hospital discharge instructions (e.g. when patients are admitted), including follow up information and how to prevent readmission, in order to improve the potential outcome of a medical condition.

In one aspect, as provided herein, implementation of a standardized disposition process may provide medicolegal oversight on scripting to ensure least amount of exposure to individuals, clinicians, and institutions. Further, in this aspect, the standardized disposition process can allow for improved dissemination of medical information, such as warning messages appropriate for a patient or guardian (e.g., influenza season, Zika virus, H1N1, etc.). In another implementation, the standardized disposition process may comprise other educational topics, for example, such as smoking cessation, medications information (e.g., antibiotics and opiates), testing and diagnostic tools (e.g., dangers of radiation) for patients.

Additionally, in one aspect, the standardized disposition methods and systems, described herein, may be used in the following service lines and/or entities, including but not limited to: Preadmission testing, pre/post-operative, general surgery, OB/Gyn, ENT, hand, urology, vascular, CT surgery, FP, IM, Peds, endocrinology, heme/oncology, plastics, ID, podiatry, maxillofacial, ortho, anesthesia, bariatrics, geriatrics, dermatology, cardiology, pulmonology, GI, PMNR, workers comp, ER, ophthalmology, neurology, pain management, radiology, trauma, endocrinology, psychiatry, psychology, hepatology, dentistry, spine, proctology, allergy and immunology, electrophysiology, pathology, neonatal, nephrology, nuclear medicine, rheumatology, sleep medicine, sports medicine, and critical care medicine.

Some examples of medical conditions that may be included in the standardized disposition methods and systems, described herein, may include:

### A:

Ankle sprain
Abdominal pain (non-specific)
Allergic reactions
Amputation (soft tissue)

### Appendicitis

Asthma exacerbation
Atrial fibrillation

### B:

Back pain/strain (thoracic, lumbar, sacral, coccyx)
Bee stings
Bell's palsy
Blood transfusion

### Bronchitis/bronchospasm

### Burns

### C:

### Cellulitis

Cervical sprain
Cervicitis (GC, chlamyia, BV/Trich)

### Chest Pain

### CHF

### Chronic Pain mgt in the ER

Closed head injury (with concussion, without concussion)

### COPD

### Cough

### Croup

### Corneal Abrasion

### Conjunctivitis

### Cat bit

### D:

### Dehydration

Dental fracture
Dental pain/abscess
Dermatitis (allergic vs. contact vs. non-specific)

### Diabetes

### Diverticulitis

### Diverticulosis

Diarrhea (non-specific vs. presumed infectious), stool cultures

### DKA

Dog bite

### DVT

### Dyspnea

### E:

Ectopic pregnancy
Ear—foreign body removal

### Epididymitis

### Epistaxis

Eye—foreign body removal, rust ring

### F:

Fever—adults, kids, proper dosage

### Fishhook

Fractures (many)

### G:

### Gastroenteritis

GI bleed (non admission, upper vs. lower)

### Gout

### H:

### Headache

Heat exhaustion
High blood pressure

### Hyperkalemia

### I:

### Immunizations

### Influenza

### J:

### K:

Kidney stones

### L:

Lacerations (many), tissue adhesive

### M:

### Medications (?)

### MVC

Muscle spasm

### N:

Nail trephination

### Neuropathy

### O:

### Orchitis

Otitis media
Otitis externa

### Ovarian Cyst

### P:

### Panic/Anxiety

### Palpitations

### Paranychia

Peptic ulcer disease

### Pharyngitis

### PID

Pilonidal cyst

### Pneumonia

### Pregnancy

Prostate enlargement

### R:

Renal failure
Rib contusion
Rib fracture
RICE therapy
Rotator cuff tendonitis/injury

### RSV

### S:

### Sciatica

### Seizures

### Sepsis

### Shingles

### SBO

Splint care

### STDExposure

### STEMI

### Strain/sprain

Strep pharyngitis

### Stye

### Sunburn

### Syncope

### T:

Tetanus update
Throat pain (viral, bacterial)

### TIA

Tobacco cessation

### U:

Upper respiratory infection

### Ureterolithiasis

Urinary retention

### Urticaria

### UTI

### V:

Vaginal bleeding

### Vaginal Discharge

### Vertigo

### Viral Exanthem

### Vomiting

### W:

### Weakness

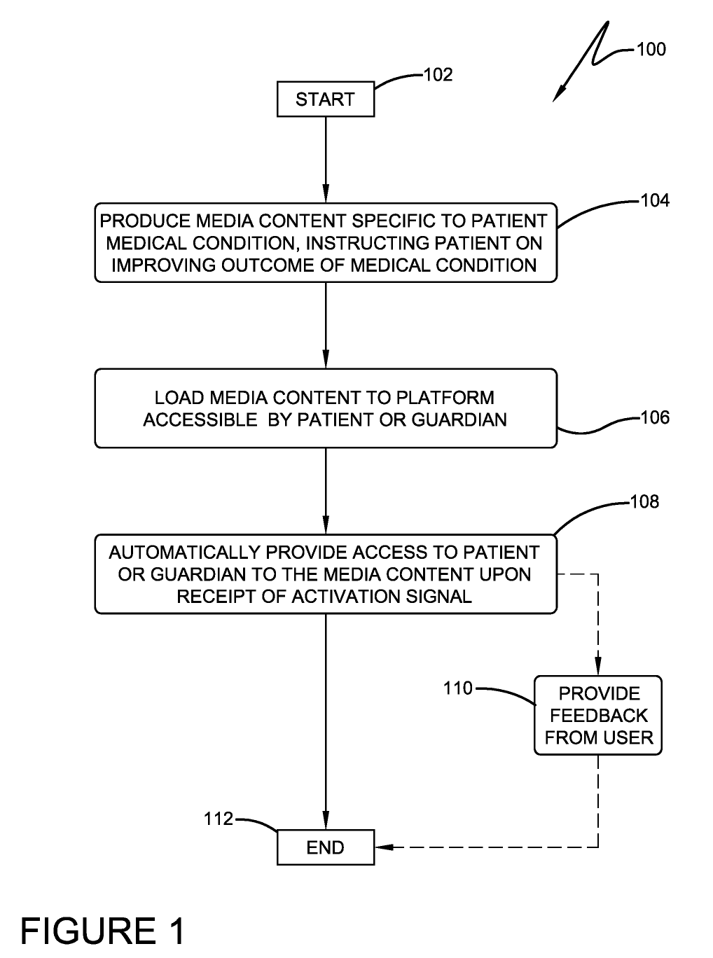

FIG. 1 is a flow diagram illustrating an example method 100 for providing disposition information to a patient. In this example implementation, the exemplary method 100 begins at 102. At 104, media content is produced, where the media content comprises patient disposition information specific to a medical condition associated with a patient. Further, in this implementation, the media content instructs the patient regarding steps to take to improve an outcome for the medical condition. For example, the media content can comprise data that is indicative of information that a patient or patient's guardian can use to help improve the outcome (e.g., improved health outcome) of the medical condition for which they are specifically receiving treatment or diagnostic analysis.

In one implementation, the medical condition can be identified as being associated with the patient based at least upon a medical code that may be found in a current version of the International Statistical Classification of Diseases (ICD) (e.g. the ICD-10). For example, the ICD is the international “standard diagnostic tool for epidemiology, health management and clinical purposes,” according to the World Health Organization (WHO), who manages the list. The ICD is a health care classification system that provides diagnostic codes for classifying diseases, including various classifications of a wide variety of signs, symptoms, abnormal indications, complaints, social circumstances, and external causes of injury or disease. The system is used to map health conditions to corresponding categories each having specific variations, with a designated code. In this implementation, for example, a code from the ICD-10 may be used to identify a corresponding media content that is specific to the code. That is, for example, a database may comprise the ICD codes, which can be linked in the database to the corresponding multi-media content that can be provided to the patient or guardian.

Further, in one implementation, the steps to take to improve outcome comprise information regarding wound care, including suture care; catheter, indwelling drain or other device instructions and care; pacemaker instructions and care; implantable device instructions and care; pre- and post-operative care and expectations after a procedure or surgery; and/or medication instructions, expectations and affects. Additional information and instructions can be provided for the patient or patient's guardian that are configured to help improve the outcome of a medical condition, such as how to treat certain conditions, when to contract your doctor, when and how to perform certain actions; situations/actions to avoid, and/or situations/actions that may improve a condition, for example. In one implementation, the content can comprise specific information and instructions that direct the patient to perform specific tasks configured to improve the outcome for the medical condition.

At 106 in the exemplary method 100, the media content is loaded to a platform that is accessible by the patient or patient's guardian. As an example, the platform can comprise an electronic medical record system, medical clinic or hospital internal network system, surgery center network, outpatient clinic network, and/or a remote medical record system. The media content can comprise data loaded and stored on a data storage system, or may comprise data loaded onto an optical disk reading system, or other media storage components that are comprised in the platform. In one implementation, the media content can comprise video and/or animated media, or other multi-media, such as images, audio, or other content.

At 108, the media content can be automatically provided on a display accessible by the patient or patient's guardian, upon the platform receiving an activation signal. In one implementation, the activation signal can be transmitted to the platform upon activation of a link provided to the patient or patient's guardian (e.g., or a healthcare provider). For example, a remote network link (e.g., URL or other link) can be provided in patient documentation (e.g., paperwork), and the patient or guardian may use the link to activate the media content. As another example, a message (e.g., email, text, social media message, other types of e-messages) comprising an activation link can be sent to the patient or guardian, which can be accessed by the patient or guardian. As another example, a mobile application (e.g., mobile app on a smart device, tablet, etc.), or a web-based application (e.g., web-app on a website or portal) may be used to access the media content. As another example, a code (e.g., bar-code, QR code, or the like) may be provided that, when scanned, directs the user (e.g., patient, guardian, healthcare provider) to the media content.

In one implementation, the media content provided to the patient or guardian at a variety of locations. For example, the media content may be provided or displayed at a hospital where the visit occurred; an ER; urgent care center; outpatient surgery center; a clinician's office or clinic; in a home health care setting; at a physical/occupational therapy center; dental clinic or surgery center; and/or a veterinary clinic. As another example, the patient or guardian may access the media content anywhere on a personal device, such as a computer, tablet, portable smart device, or the like, by accessing the content via a remote network (e.g., Internet or intranet), such as using a web-portal, web-app, website, mobile app, or other access tools.

In one implementation, at 110 in the exemplary method 100, the method may provide for user feedback, such as by the patient, guardian or health care provider. That is, of example, the media content may provide for the user to indicate that the content has been viewed (e.g., or otherwise reviewed), and/or understood (e.g., by selecting an on-screen button, or other action). As another example, the media content may provide for questions, comments, or other input to be provided. In this example, the questions may be provided to the appropriate health care provider/clinician; or comments and other input may be appropriately added to the patient's record (e.g., EMR).

In the exemplary method of FIG. 1, having automatically provided access to the media content, to the patient or guardian (e.g., and provided for user feedback), the method 100 ends at 112.

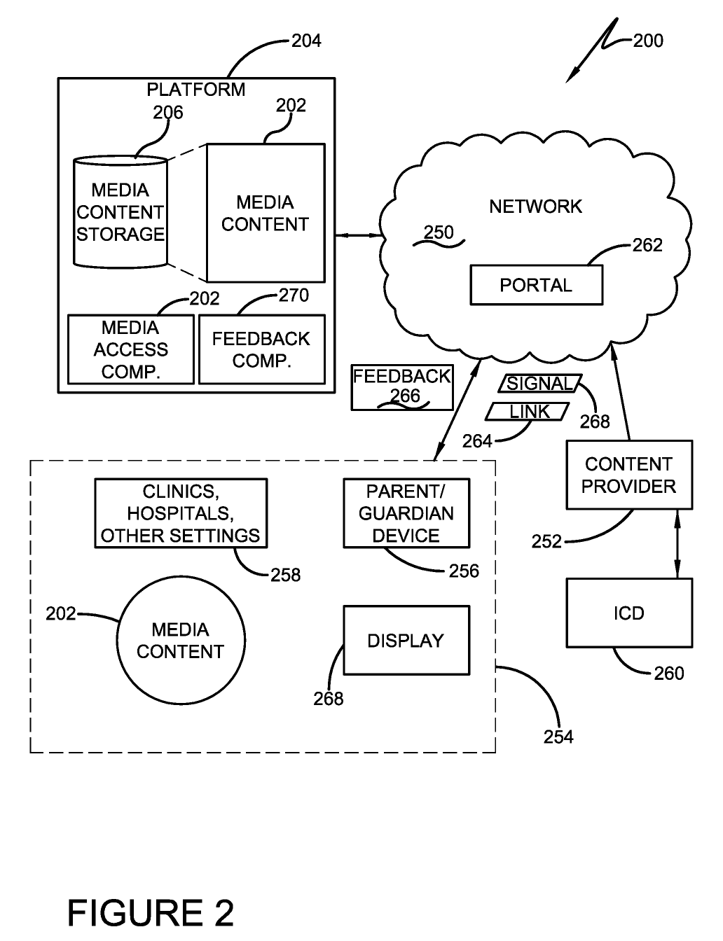

In another aspect, a system may be devised that facilitates medical disposition for a patient. FIG. 2 is a schematic diagram of an exemplary system 200 for providing medical disposition. In this example system 200, media content 202 can comprise patient disposition information that is specific to a medical condition associated with a patient 256. In this implementation, the media content 202 can instruct the patient regarding steps to take to improve an outcome for the medical condition. Further, in this system, a platform 204 can provide access to the media content 202. The platform 204 can comprise a media content storage component 206 that stores the media content 202 (e.g., electronically, or otherwise). The platform 204 can also comprise a media access component 208 that automatically provides access to the media content 202 by the patient of patient's guardian 256 on a display 268 upon the platform 204 receiving an activation signal 268 authorizing access to the media content 202.

In one implementation, the medical condition identified as being associated with the patient can be base, at least, upon a medical code found in a current version of the International Statistical Classification of Diseases (ICD) 260. For example, a provider 252 (e.g., creator, producer) of the media content may be able to link specific medical conditions with the appropriate ICD code, such as in a database on the platform 204.

In one implementation, the display 268 that provides access to the media content 202 can be disposed at a variety of locations 254, for example, remotely linked to a network 250, that is linked or comprises the platform 204. For example, the location can comprise hospitals, clinics, clinician offices, homes of patient, etc. 258; and/or an location that the patient or guardian 256 may be able to access the network 250, or otherwise view the media content 202 (e.g., on an optical disc or loaded onto a portable storage). In one implementation, the media content 202 can comprise video and/or animated media (e.g., or other multi-media content) that comprise specific information and instructions that direct the patient to perform specific tasks related to improving the outcome for the medical condition.

In one implementation, the media access component 208 can comprise (e.g., or be linked to, such as through the network 250) a web-portal 262. The web-portal can comprise a website on the Internet, for example, a specific location on a private network (e.g., intranet), an application, mobile application, or other web-app. In this example, the patient or guardian may be able to access the portal 262 remotely or locally, using a personal device, or one provided by the clinic/hospital (e.g., or by healthcare provider). In one implementation, access to the media content 202 (e.g., or web-portal) can be provide by a link 264, such as accessible from documentation provided to the patient or guardian, a message sent to the patient or guardian, a mobile app, a code that can be scanned, and/or a web-app.

In one implementation, the example system 200 can comprise a patient feedback component 270 that receives feedback 266 from the patient or patient's guardian 256 regarding acceptance of the media content 202, comments regarding the media content 202, and/or questions regarding the media content 202. As an example, the feedback 266 may be used to update the patient medical records, and/or allow a healthcare provider to determine that appropriate media content has been provided to the patient 256.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Of course, those skilled in the art will recognize many modifications may be made to this configuration without departing from the scope or spirit of the claimed subject matter.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure.

In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. A method for medical disposition of a patient, comprising:
producing media content comprising patient disposition information specific to a medical condition associated with a patient, the media content instructing the patient regarding steps to take to improve outcome for the medical condition;
loading the media content to a platform accessible by the patient or patient's guardian;
automatically providing the media content on a display accessible by the patient or patient's guardian upon the platform receiving an activation signal.

2. The method of claim 1, the medical condition identified as being associated with the patient based at least upon a medical code found in a current version of the International Statistical Classification of Diseases (ICD).

3. The method of claim 1, the steps to take to improve outcome comprising one or more of:
wound care;
catheter, indwelling drain or device instructions;
pacemaker care,
implantable device care,
pre and post-operative care and expectations after procedure or surgery; and
medication instructions and affects.

4. The method of claim 1, the media content provided to the patient or guardian at one of:
a hospital;
ER;
urgent care;
outpatient surgery center;
clinician's clinic or office,
home health provision area;
physical or occupational therapy center;
dental clinic or surgery center;
veterinary clinic;
optometry clinic or office; and
other patient care areas.

5. The method of claim 1, the platform comprising one or more of:
an electronic medical record system;
medical clinic or hospital internal network system;
surgery center network;
outpatient clinic network; and
remote medical record system.

6. The method of claim 1, the media content comprising video and/or animated media comprising specific information and instructions that direct the patient to perform specific tasks related to improving the outcome for the medical condition.

7. The method of claim 1, the media content accessible by the patient or patient's guardian through a web-based portal.

8. The method of claim 1, the activation signal transmitted to the platform upon activation by one of:
use of a remote network link provided in patient documentation;
use of a link provided in a message sent to the patient or patient's guardian;
a mobile application;
scanning of a code by a network capable device; and
a web-based application.

9. The method of claim 1, comprising receiving feedback from the patient or patient's guardian regarding acceptance of the media content, comments regarding the media content, and/or questions regarding the media content.

10. A medical disposition system comprising:
media content comprising patient disposition information specific to a medical condition associated with a patient, the media content instructing the patient regarding steps to take to improve outcome for the medical condition; and
a platform that provides access to the media content, the platform comprising:
a media content storage component that stores the media content; and
a media access component that automatically provides access to the media content by the patient of patient's guardian on a display upon the platform receiving an activation signal authorizing access to the media content.

11. The system of claim 10, the medical condition identified as being associated with the patient based at least upon a medical code found in a current version of the International Statistical Classification of Diseases (ICD).

12. The system of claim 10, the steps to take to improve outcome comprising information on one or more of:
wound care;
catheter, indwelling drain or device;
pacemaker care,
implantable device care,
pre and post-operative care and expectations after procedure or surgery; and
medication and affects.

13. The system of claim 10, the display providing access to the media content disposed at one of:
a hospital;
ER;
urgent care;
outpatient surgery center;
clinician's clinic or office,
home health provision area;
physical or occupational therapy center;
dental clinic or surgery center;
veterinary clinic;
optometry clinic or office; and
other patient care areas.

14. The system of claim 10, the platform comprising one or more of:
an electronic medical record system;
medical clinic or hospital internal network system;
surgery center network;
outpatient clinic network; and
remote medical record system.

15. The system of claim 10, the media content comprising video and/or animated media comprising specific information and instructions that direct the patient to perform specific tasks related to improving the outcome for the medical condition.

16. The system of claim 10, the media access component comprising a web-based portal.

17. The system of claim 10, the activation signal transmitted to the platform upon activation by one of:
use of a remote network link linking to the media access component provided in patient documentation;
use of a link linking to the media access component provided in a message sent to the patient or patient's guardian;
a mobile application linking to the media access component;
scanning of a code by a network capable device linking to the media access component; and
a web-based application linking to the media access component.

18. The system of claim 10, comprising a patient feedback component that receives feedback from the patient or patient's guardian regarding acceptance of the media content, comments regarding the media content, and/or questions regarding the media content.

19. A system for providing medical disposition instruction for a patient, comprising:
media content comprising patient disposition information specific to a medical condition associated with a patient by a medical code found in a current version of the International Statistical Classification of Diseases, the media content utilizing video and/or animated media comprising specific information and instructions that direct the patient to perform specific tasks related to improving the outcome for the medical condition; and
a platform liked to an electrical medical record that provides access to the media content, the platform comprising:
a media content storage component that stores the media content remotely from a patient location; and
a media access component that receives an activation signal from an action performed by the patient or patient's guardian to automatically provides access to the media content on a display.

20. The system of claim 10, comprising a patient feedback component that receives feedback from the patient or patient's guardian regarding acceptance of the media content, comments regarding the media content, and/or questions regarding the media content.
